(19) United States
(12) Patent Application Publication
(10) Pub. No.: US 2005/0042202 A1
(43) Pub. Date: Feb. 24, 2005

(54) Compositions for and methods of treating and preventing sirs/sepsis

(76) Inventors: David B. Weiner, Merion Station, PA (US); Karuppiah Muthumani, Cherry Hill, NJ (US)

Correspondence Address: COZEN O'CONNOR, P.C., 1900 MARKET STREET, PHILADELPHIA, PA, 19103-3508, US

(21) Appl. No.: 10/491,800

(22) PCT Filed: Oct. 7, 2002
(86) PCT No.: PCT/US02/32084

Related U.S. Application Data
(60) Provisional application No. 60/327,270, filed on Oct. 5, 2001.

(51) Int. Cl. A61K 48/00; A61K 38/17

(52) U.S. Cl. 424/93.2; 514/12; 514/44

(57) ABSTRACT

The present invention relates to methods of inhibiting the immune response underlying sepsis, to methods of preventing sepsis, to methods of treating sepsis and to pharmaceutical compositions useful in such methods.

### FIELD OF THE INVENTION

The present invention relates to methods of inhibiting the immune response underlying SIRS/sepsis, to methods of preventing SIRS/sepsis, to methods of treating SIRS/sepsis, and to pharmaceutical compositions useful in such methods.

### BACKGROUND OF THE INVENTION

Bacterial infection and other potent stimuli initiate immune responses that can result in systemic inflammation, or (systemic inflammatory response syndrome) (SIRS) (Bumell R., 1994, Circ. Shock 43:137; Bahrani S, et al., 1995, Prog. Clin. Biol. Res. 392:197; incorporated by reference herein). Severe SIRS will lead to “multiple organ failure syndrome” (MODS), which involves varying degrees of fever, hypotoxemia, trachypnea, tachycardia, endothelial inflammation, myocardial insufficiency, hyperfusion, altered mental status, vascular collapse, and ultimately organ damage such as acute respiratory distress syndrome, coagulopathy, cardiac failure, renal failure, shock, and/or coma. (American College of Chest Physicians/Society of Critical Care Medicine Consensus Conference, Crit. Care Med., 1992, 20:864; and Bone, 1995, JAMA 273:155, which are each incorporated herein by reference).

When SIRS is caused by infection, it is termed sepsis. Sepsis can progress into the clinical stages of severe sepsis, and ultimately septic shock. Clinical sepsis is broadly defined to mean situations where the invasion by a microbial agent is associated with the clinical manifestations of infection. The clinical symptoms of SIRS and sepsis include, but not limited to, (1) temperature>38° C. or <36° C.; (2) heart rate of >90 beats per minute; (3) respiratory rate>20 breathes per minute or PaCO₂<32 mm Hg; (4) white blood cell count>12000/cu mm, <4,000/cu mm, or >10% immature (band) forms; (5) organ dysfunction, hyperfusion, or hypertension (Bone et al., 1992, Chest 101:1644, which is incorporated by reference herein). Septic shock is characterized by inadequate tissue perfusion, leading to insufficient oxygen supply to tissues, hypotension and olgiuria.

When an infection occurs, macrophages at the site of infection are activated to secrete TNF-a and IL-1, leading to increased release of plasma proteins into tissue, increased phagocyte and lymphocyte migration into tissue, and increased platelet adhesion to the blood vessel wall. In this way, the local vessel is occluded and the pathogen is localized to the site of infection. However, in sepsis, the infection becomes systemic and the TNF-a induced blood vessel occlusion becomes catastrophic. The systemic release of TNF-a causes vasodilation and loss of plasma volume due to increased vascular permeability, leading to shock. In septic shock, TNF-a further triggers disseminated intravascular coagulation (blood clotting) leading to the generation of clots in the small vessels and the massive consumption of clotting proteins. As the patient's ability to clot blood is lost, vital organs such as kidneys, liver, heart, and lungs are compromised due to failure of normal perfusion. Septic shock results in a mortality rate as high as 81%.

Escherichia coli is the infecting pathogen in many cases of sepsis, however, other gram-negative bacteria, such as those of the Klebsiella-Enterobacter-Serratia group and Pseudomonas may also initiate the condition. Gram-positive microbes such as Staphlococcus, and systemic viral and fungal infections also initiate sepsis in a small number of cases. The genitourinary tract, gastrointestinal tract, and respiratory tract are the most common sites of infection leading to sepsis. Other sepsis-related sites of infection include wound, burn and pelvic infections, and infected intravenous catheters.

Sepsis is most common in hospitalized patients having underlying diseases or conditions that render them susceptible to bloodstream invasion, or in burn, trauma or surgical patients. Factors that render a patient susceptible to bloodstream invasion include a generally weakened immune system, such as found in neonates and the elderly, and a condition or disease that results in a increased local susceptibility to infection, such as impaired circulation, diabetes, uremia and AIDS. Finally, subjects with a propensity for exaggerated immune response, such as may occur due to the presence of certain alleles of the IL-1 gene, are also at higher risk to develop sepsis (U.S. Pat. No. 6,251,598, which is incorporated herein by reference).

Since the prognosis of patients with sepsis is not dependent on the severity of the infection, but on the severity of the SIRS/septic reaction of the patient (Pilz et al., 1991, Krankenpflege-Journal 29:483, which is incorporated herein by reference), many diagnostic assays focus on aspects of the immunological response. Centocor Inc. 's immunometric assay for TNF-a (WO 90/06314, which is incorporated herein by reference) uses two antibodies to measure the level of this inflammatory mediator in a patient's blood. The assay developed by the National Aeronautics and Space Administration detects Pseudomonas bacteria in a patient's blood by extraction of Azurin, and its detection by a monoclonal antibody (U.S. Pat. No. 5,210,019, which is incorporated herein by reference). The sepsis assay from BioWhittaker (Walkerville, Md.) and the Limulus Amebocyte Lysate Assay form Seikagaku Kyoto Ltd. (Tokyo, Japan) measure levels of E. coli endotoxin in a patient's blood. Other sepsis diagnostic assays detect the presence of oxidants produced by white blood cells during the septic response (U.S. Pat. No. 5,804,370, which is incorporated herein by reference), the sepsis marker peptide procalcitonin (U.S. Pat. No. 5,639,617, which is incorporated herein by reference), leukocyte high-affinity Fc receptor (CD64) expression (Davis et al., 1995, Laboratory Hematology 1:3, which is incorporated herein by reference); serum C-reactive protein (CRP) (Drews et al., 1995, Ann. NY Acad. Sci. 762:398; Kawamura and Nishida, 1995, Acta Paediatr. 84:10 which are both incorporated herein by reference); neurophil surface CD11b levels (U.S. Pat. No. 6,077,665, which is incorporated herein by reference), and the ratio of selected unsaturated free fatty acids to saturated free fatty acids (U.S. Pat. No. 5,780,237, which is incorporated herein by reference).

The HIV-1 accessory gene vpr encodes viral protein R (Vpr) which has been implicated in the regulation of many host cellular events including proliferation, differentiation, apoptosis, cytokine production, and NF-?B mediated transcription (Levy et al., 1993, Cell 72:541-550; Ayyavoo et al., 1997, Nature Medicine 3:1117-1123; Stewart et al., 1997, J. Virol. 71:5579-5592.) NF-?B activation is important for the induction of some cytokines and chemokines which specifically expand antigen specific immune responses. NF-?B activation also plays an important role in the induction of proinflammatory cytokines, in particular TNFa, triggered through the CD28 costimulatory pathway (Moriuchi et al., 1997, J. Immunol. 158:3483-3491; Fraser et al., 1992, Mol. Cell. Biol. 12:4357-4363.) The pattern of cytokine expression influences the nature and persistence of the inflammatory response. For instance, production of IFN-? and TNF are well-suited to induce enhanced cellular immunity, while TL-4 and IL-10 are associated with helping B cells develop into antibody-producing cells (Paul et al., 1994, Cell 76:241-251). Studies using mutant NF-?B binding sites and Ik? a competition have shown that transcription factors including NF-?B and SP-1 are important for RANTES (Regulated on Activation, Normal T Expressed and Secreted) gene expression (Moriuchi et al., 1997, J. Immunol. 158:3483-3491.)

CD8⁺ T cells are believed to play an important role in controlling HIV infection through CTL induction. Additionally, CD8⁺ T cells are involved in secretion of several factors including the 13-chemokines RANTES, MIP-1a, MIP-1β, and MDC (Brinchman et al., 1990, J. Immunol. 114:2961-2966; Cocchi et al., 1996, Science 270:1811-1815; Pal et al., 1997, Science 278:695-698). CD8⁺ CTL is an important immunological defense against viral infections.

Chemokines are important for the regulation of lymphocyte recruitment in infection and immune activation (Schall et al., 1994, Curr. Opin. Immunol. 6:865-873.) T cell activation results in synthesis of certain chemokines/cytokines which are necessary for antigen-specific T helper cell as well as for cytotoxic effector cell expansion (Weiss et al., 1994, Cell 76:263-274). In addition to their role in T cell trafficking and immune activation, the β-chemokines can inhibit HIV-1 infection in established macrophage cell lines as well as in primary lymphocytes through interference with viral coreceptors required for entry (Feng et al., 1996, Science 272: 872-877; Dornaz et al., 1996, Cell 85:1149-1158). For example, chemokines are produced by some subsets of T cells following T cell receptor (TCR) and CD28/CTLA-4 co-ligation (Taub et al., 1996, J. Immunol. 156:2095-2103; Herold et al., 1997, J. Immunol. 159:4150-4153).

Induction of T cell proliferation, CTL induction and cytokine secretion each requires both the engagement of the TCR complex and interaction of the CD28/CTLA-4 costimulatory molecules with their ligands CD80 or CD86 present on antigen presenting cells (APCs) or through the CD40/CD40 ligand providing the required second signal to induce T cell activation (Fraser et al. 1994, Immunol. Today 14:357-362; Crabtree et al., 1989, Science 243:355-361; Linsley et al., 1993, Annu. Rev. Immunol. 11:191-212; June et al., 1994, Immunol. Today 15:321-331.) Studies have shown that T cell activation through CD28 enhances production of B-chemokines, yielding anti-viral effects (Carroll et al., 1997, Science 276:273-276; Levine et al., 1996, Science 272:1939-1943; Bisset et al., 1997, AIDS 11:485-491.) Recruitment and activation of CD8⁺ cells at the site of inflammation increases specific CTL precursor frequency (Stevenson et al., 1997, Eur. J. Immunol. 27:3259-3268; Doherty et al., 1997, Immunol. Reviews 159:105-117). Blocking the synthesis of chemokines may ameliorate the symptoms of inflammatory diseases which rely on the synthesis of chemokines to recruit cells responsible for the immune response.

Cellular immunity, specifically the MHC-restricted CTL response is thought to play an intrinsic role in protection and clearance of a number of viral infections. Reduction in the number of CD8⁺ T cells in HIV-1 infected individuals has been correlated with reduced anti-viral effect and disease progression in parallel with the deterioration of the immune system (Mackewicz et al., 1991, J. Clin. Invest. 87 1462-1466; Pantaleo et al., 1997, Proc. Natl. Acad. Sci. USA 949848-9853.) Information from studies on HIV-1 infected long-term non-progressors, uninfected infants born to HIV-1 infected mothers, and seronegative individuals repeatedly exposed but as yet uninfected have supported the role of CTL responses in controlling viral load and perhaps even limiting disease progression (Borrow et al., 1994, J. Virol. 68:6103-6110; Rowland-Jones et al., 1995, Nature Medicine 1:59-94.)

There remains a need for methods and pharmaceutical compositions to treat systemic inflammation, such as SIRS, and more specifically sepsis and sepsis shock. Such methods are particularly needed to prevent SIRS/sepsis in patients at risk of developing the condition, and effectively treating patients that are diagnosed with SIRS/sepsis.

### SUMMARY OF THE INVENTION

The invention relates to a method of treating an individual who has been diagnosed as having SIRS or sepsis comprising the step of administering to said individual a therapeutically effective amount of an immuno-modulating pharmaceutical composition comprising one or more of the following components: Vpr protein; a function fragment of Vpr protein; a nucleic acid encoding Vpr protein operably linked to regulatory elements; and a nucleic acid encoding a functional fragment of Vpr protein operably linked to regulatory elements. Preferably, the step of administering the immuno-modulating pharmaceutical composition is repeated at least once, and more preferably 1 to 6 times a day. In another preferred embodiment, the step of administering the immuno-modulating pharmaceutical composition comprises continuous administration.

In some embodiments of the method of treating an individual who has been diagnosed as having SIRS or sepsis, the individual is administered a nucleic acid encoding Vpr protein or functional fragment thereof operably linked to regulatory elements. The nucleic acid is preferably administered in a dose of 1 to 500 micrograms nucleic acid, 25 to 250 micrograms nucleic acid or about 100 micrograms nucleic acid. The nucleic acid encoding Vpr protein or functional fragment thereof operably linked to regulatory elements is preferably contained in a plasmid or in a viral vector. The viral vector is preferably a retroviral vector and an adenoviral vector.

In some embodiments of the method of treating an individual who has been diagnosed as having SIRS or sepsis, the individual is administered Vpr protein or a functional fragment thereof. Preferably, the Vpr protein or functional fragment thereof is administered at 0.1 to 100 mg/kg body weight per day, 0.5 to 50 mg/kg body weight per day or 1.0 to 10 mg/kg body weight per day.

In other embodiments, the aforementioned methods of treating an individual who has been diagnosed as having SIRS or sepsis additionally comprise at least one step of administering to said individual a therapeutically effective amount of an anti-infective agent. The anti-infective agent is preferably amikacin, tobramycin, netilmicin, gentamicin, cephalosporin, ceftazidime, maxalactam, carbopenem, imipenem, aztreonam; ampicillin, penicillin, ureidopenicillin, augmentin, amphotericin, famvir or acyclovir. The step of administering the anti-infective agent is preferably performed at the same time as the step of administering the immuno-modulating pharmaceutical preparation.

In other embodiments, the aforementioned methods of treating an individual who has been diagnosed as having SIRS or sepsis comprise the additional steps of monitoring the concentration of pro-inflammatory cytokines and sepsis marker proteins/conditions in the blood plasma of the individual, determining the need for additional doses of the immuno-modulating pharmaceutical composition and administering additional doses the immuno-modulating pharmaceutical composition. Preferably, the blood plasma level of TNFa is monitored and determining the need for additional doses of the immuno-modulating pharmaceutical composition is determined by a blood plasma level of TNFa above about 25 pg/ml.

Another aspect of the invention is a method of preventing SIRS/sepsis in an individual who has been identified as being at an elevated risk of contracting SIRS/sepsis comprising the step of administering to said individual a therapeutically effective amount of an immuno-modulating pharmaceutical composition comprising one or more of the following components: Vpr protein; a function fragment of Vpr protein; a nucleic acid encoding Vpr protein operably linked to regulatory elements; and a nucleic acid encoding a functional fragment of Vpr protein operably linked to regulatory elements. Preferably the step of administering the immuno-modulating pharmaceutical composition is repeated at least once, and more preferably 1 to 6 times a day. Preferably, the step of administering the immuno-modulating pharmaceutical composition comprises continuous administration.

In some embodiments of the method of preventing SIRS/sepsis in an individual who has been identified as being at an elevated risk of contracting SIRS/sepsis, the individual is administered a nucleic acid encoding Vpr protein or functional fragment thereof operably linked to regulatory elements. The nucleic acid is preferably administered in a dose of 1 to 500 micrograms nucleic acid, 25 to 250 micrograms nucleic acid or about 100 micrograms nucleic acid. The nucleic acid encoding Vpr protein or functional fragment thereof operably linked to regulatory elements is preferably contained in a plasmid or in a viral vector. The viral vector is preferably a retroviral vector and an adenoviral vector.

In some embodiments of the method of preventing SIRS/sepsis in an individual who has been identified as being at an elevated risk of contracting SIRS/sepsis, the individual is administered Vpr protein or a functional fragment thereof. Preferably, the Vpr protein or functional fragment thereof is administered at 0.1 to 100 mg/kg body weight per day, 0.5 to 50 mg/kg body weight per day or 1.0 to 10 mg/kg body weight per day.

In other embodiments, the aforementioned methods of preventing SIRS/sepsis in an individual who has been identified as being at an elevated risk of contracting SIRS/sepsis additionally comprise at least one step of administering to said individual a therapeutically effective amount of an anti-infective agent. The anti-infective agent is preferably amikacin, tobramycin, netilmicin, gentamicin, cephalosporin, ceftazidime, maxalactam, carbopenem, imipenem, aztreonam; ampicillin, penicillin, ureidopenicillin, augmentin, amphotericin, famvir or acyclovir. The step of administering the anti-infective agent is preferably performed at the same time as the step of administering the immuno-modulating pharmaceutical preparation.

In other embodiments, the aforementioned methods of preventing SIRS/sepsis in an individual who has been identified as being at an elevated risk of contracting SIRS/sepsis comprise the additional steps of monitoring the concentration of pro-inflammatory cytokines and sepsis marker proteins/conditions in the blood plasma of the individual, determining the need for additional doses of the immuno-modulating pharmaceutical composition and administering additional doses the immuno-modulating pharmaceutical composition. Preferably, the blood plasma level of TNFa is monitored and determining the need for additional doses of the immuno-modulating pharmaceutical composition is determined by a blood plasma level of TNFa above about 25 pg/ml.

Another aspect of the invention is a pharmaceutical composition useful for preventing and treating sepsis, comprising an anti-infective agent and one or more of the components selected from the group consisting of: Vpr protein; a function fragment of Vpr protein; a nucleic acid encoding Vpr protein operably linked to regulatory elements; and a nucleic acid encoding a functional fragment of Vpr protein operably linked to regulatory elements. Preferably, the anti-infective agent is selected from the selected from the group consisting of amikacin, tobramycin, netilmicin, gentamicin, cephalosporin, ceftazidime, maxalactam, carbopenem, imipenem, aztreonam; ampicillin, penicillin, ureidopenicillin, augmentin, amphotericin, famvir and acyclovir. Preferably, the pharmaceutical composition additionally comprises at least one adjunctive agent in the treatment of SIRS/sepsis.

Another aspect of the invention is a pharmaceutical composition useful for preventing or treating sepsis, comprising a prophylactic or therapeutically effective amount of one or more of the components selected from the group consisting of: Vpr protein; a function fragment of Vpr protein; a nucleic acid encoding Vpr protein operably linked to regulatory elements; and a nucleic acid encoding a functional fragment of Vpr protein operably linked to regulatory elements. In some embodiments, the pharmaceutical compositions are formulated for continuous infusion. In some embodiments, the pharmaceutical compositions are formulated for divided doses.

### DESCRIPTION OF PREFERRED EMBODIMENTS OF THE INVENTION

I. Definitions

As used herein, the terms “protein” and “polypeptide” are used interchangeably and intended to refer to proteinaceous compounds including proteins, polypeptides and peptides.

As used herein, the term “individual” refers to the vertebrate targeted for use of the present invention. Examples of “individuals” contemplated by the present invention include but are not limited to humans, higher order primates, canines, felines, bovines, equines, ovines, porcines, avians, and other mammals.

As used herein, the term “administration” refers to the delivery of polypeptides to an individual. “Administration” also refers to the delivery of nucleic acids which encode polypeptides. The term includes, but is not limited to delivery routes including orally, intramuscularly, intravenously, intranasally, intraperatoneally, intradermally, intrathecally, intraventricularly, subcutaneously, transdermally, topically or by lavage. Modes of administration contemplated by this invention include but are not limited to the use of a syringe, intravenous line, transdermal patch, or needleless injector.

As used herein, functional fragments of Vpr are fragments of Vpr which have the ability to attenuate immune responses in an individual or cell culture. A functional fragment of Vpr can be determined in assays the measure the effect of the Vpr fragment on the production of beta chemokines, such as MIP-1a, MIP-Iβ and RANTES, in human macrophages and primary lymphocytes (Muthumani K, et al., 2000, J. Leukoc. Biol., 68, 366-372, incorporated by reference herein). Fragments of Vpr that decrease the production of beta chemokines in human macrophages or primary lymphocytes are considered functional fragments of Vpr with respect to the present invention. Alternatively, functional fragments may be determined by the presence of the functional domains necessary for the nuclear localization and cell cycle arrest activities of Vpr (Mahalingam, S, et al., 1997, J. Virol., 71, 6339-6347; Macreadie, I. G. et al., 1995, Proc. Natl. Acad. Sci., 92, 2770-2774; both of which are incorporated by reference herein). Generally, functional fragments comprise a length of amino acid sequence from Vpr that is more that 5 amino acids in length, preferably more than 10 amino acids in length, and most preferably more than 20 amino acids in length. In some embodiments, the functional fragments comprise a 5-30 residue fragment of the 30 most amino residues of the amino terminal sequences of Vpr. In some embodiments, the functional fragments comprise a 5-30 residue fragment of the 30 most carboxy residues of the carboxy terminal sequences of Vpr

As used herein, the treatment of SIRS/Sepsis refers to the reduction amelioration or elimination of clinical symptoms associated with SIRS and Sepsis and those conditions associated with MODS such as varying degrees of fever, hypotoxemia, trachypnea, tachycardia, endothelial inflammation, myocardial insufficiency, hyperfusion, altered mental status, vascular collapse, and ultimately organ damage such as acute respiratory distress syndrome, coagulopathy, cardiac failure, renal failure, shock, and/or coma.

### II. DESCRIPTION OF THE PREFERRED EMBODIMENTS OF THE INVENTION

The present invention provides improved methods to prevent (systemic inflammation response syndrome) (SIRS) in individuals at risk of developing such an condition, and methods to treat individuals who have developed SIRS. The SIRS prevention and treatment methods employ one of the weapons that the HIV virus uses to evade and undermine an infected individual's immune system: the Vpr protein and/or a nucleic acid molecule that encodes it. Armed with this HIV-derived weapon, sepsis prevention and treatment can be made more effective by reducing the overstimulation of the individual's immune response that is the immediate cause of SIRS. Moreover, the present invention uses the HIV Vpr protein and/or a nucleic acid molecule that encodes it to treat individuals who have sepsis, or at risk of developing sepsis.

The present invention arises from the surprising discovery that the delivery of Vpr polypeptide suppresses cellular immune responses including those associated with SIRS and Sepsis. While not limiting the invention to any one mechanism, Vpr inhibits the synthesis of protypic Th1 type cytokines, including TNF-a, and shifts the antibody response toward a Th2 type bias. Moreover, Vpr suppresses CC chemokines and compromises CD8⁺ T Cell Effector function. The data support the conclusion that Vpr interferes with costimulatory molecules involved in immune activation. Accordingly, when delivered to an individual with SIRS/sepsis, or who is at risk of developing SIRS/sepsis, Vpr attenuates the immune response that leads to systemic TNFa release. When delivered to an individual who has sepsis, Vpr attenuates the systemic immune response that leads ultimately to septic shock. Particularly surprising, it has been found that once effective treated for Sepsis, the methods of the present invention prevent and protect against recurrence or subsequent incidents of SIRS/Sepsis.

The amino acid sequence of Vpr and the DNA sequence that encodes it are described in U.S. Pat. No. 5,874,225 issued on Feb. 23, 1999, which is incorporated herein by reference, including the patents and publications referred to therein. Fragments of Vpr are described in PCT/US94/02191 filed Feb. 22, 1994, PCT/US95/12344 filed Sep. 21, 1995, and PCT/US98/16890 filed Aug. 14, 1998, which are each incorporated herein by reference together with the respective corresponding U.S. National Stage applications claiming priority thereto, and U.S. Pat. No. 5,763,190 issued Jun. 9, 1998, which is incorporated herein by reference. U.S. Ser. No. 08/167,608 filed Dec. 15, 1993 and PCT/US94/00899 filed Jan. 26, 1994, which are incorporated herein by reference, describe recombinant viral particles which include functional fragments of Vpr protein as part of the viral particle. Mutational analysis of the Vpr protein has identified distinct functional domains required for the nuclear localization and cell cycle arrest activity of Vpr (Mahalingam, S, et al., 1997, J. Virol., 71, 6339-6347; Macreadie, I. G. et al., 1995, Proc. Natl. Acad. Sci., 92, 2770-2774; both of which are incorporated by reference herein).

A. Methods to Prevent SIRS/Sepsis

The present invention provides a method to prevent SIRS in an individual who is identified as being at an elevated risk of contracting SIRS. In a preferred embodiment, the individual is identified as being at an elevated risk of developing sepsis. According to the invention, methods of treating an individual at risk of developing an excessive systemic immune response comprise administering to the individual a prophylactically effective amount of Vpr protein, as Vpr protein or a functional fragment thereof, or as a nucleic acid encoding Vpr protein or a functional fragment thereof, or a combination of two or more of the same. When a nucleic acid encoding Vpr protein or a functional fragment thereof is delivered to an individual, the coding sequence is operably linked to regulatory elements. Once delivered to the individual, the nucleic acid encoding the Vpr protein is expressed and the Vpr protein is synthesized within the individual. In some embodiments, the Vpr is delivered as a nucleic acid molecule with the coding sequence for Vpr protein and/or a functional fragment thereof. In some embodiments, the Vpr and/or a functional fragment thereof is delivered as a protein. Once delivered to the individual, the presence of the Vpr protein, either delivered as a protein or produced by the expression of the nucleic acid molecule that encodes it, inhibits the undesirable immune response.

In another embodiment, the method to prevent SIRS/sepsis comprises the additional steps of monitoring the concentration of pro-inflammatory cytokines and sepsis marker proteins/conditions in the blood plasma of the individual, determining if the monitored cytokine/marker indicates the patient has SIRS/sepsis, and administering additional doses of Vpr protein and/or functional fragments thereof, and/or nucleic acids encoding the Vpr protein and/or fragment thereof. Pro-inflammatory cytokines and sepsis marker proteins/conditions suitable for monitoring include, but are not limited to, cytokines such as TNFa, IL-1, IL-6, IL-8, IL-12; chemokines such as IL-8, PBP, β-TG, NAP-2, GROa, GROβ, GRO?, IP-10, SDF-1, MIP-1a, MIP-1β, MCP-1, RANTES, eotaxin, lymphotactin, fratalkine; and marker proteins/conditions such as Azurin, E. Coli and other Gram-negative bacteria endotoxins, oxidants produced by white blood cells, procalcitonin, leukocyte high-affinity Fc receptor (CD64); serum C-reactive protein (CRP); neurophil surface CD11b levels, and the ratio of selected unsaturated free fatty acids to saturated free fatty acid. In a preferred embodiment, the concentration of TNF-a in the blood plasma is monitored. In preferred embodiments, a individual is determined to have SIRS/sepsis when the concentration of TNF-a in the blood plasma is above about 100 pg/ml, more preferably above about 50 pg/ml, most preferably above about 25 pg/ml.

Individuals at an elevated risk for SIRS/sepsis include, but are not limited to, subjects with a propensity for exaggerated immune response, such as may occur due to the presence of certain alleles of the IL-1 gene (U.S. Pat. No. 6,251,598, which is incorporated by reference herein). Individuals at an elevated risk for sepsis include, but are not limited to, individuals, particularly those hospitalized, having underlying diseases or conditions that render them susceptible to bloodstream invasion, or burn, trauma, wound or surgical patients. Factors that render a patient susceptible to bloodstream invasion include, but are not limited to, a generally weakened immune system, such as found in neonates or the elderly, or a condition or disease that results in a increased local susceptibility to infection, such as impaired circulation, diabetes, and uremia.

The phrase “prophylactically effective amount” is used herein to mean an amount sufficient to prevent the onset of symptoms associated with SIRS/sepsis in an individual at risk of developing SIRS/sepsis. The prophylactically effective amount preferably reduces by at least about 30 percent, more preferably by at least 50 percent, most preferably by at least 90 percent, a clinically significant increase in the blood plasma level of TNFa. A clinically significant increase in the plasma level of TNFa is an increase to above about 25 pg/ml blood plasma, preferably above about 50 pg/ml blood plasma, and most preferably above about 100 pg/ml blood plasma. Methods for determining the plasma TNFa levels are well known in the art, (U.S. Pat. No. 6,168,790; U.S. Pat. No. 6,063,764; Creasey et al., 1991, Circ. Shock 33:84-9; which are incorporated by reference herein)

It should be noted that levels of TNFa in normal healthy humans or in laboratory animals are estimated to be no more than about 10 pg/ml (Michie, et al., New Eng J. Med., 318:1481-1486, 1988; Mathison, et al., J. Clin. Invest., 81:1925, 1988; and Waage, et al., Lancet, 1:355-357,1987; incorporated by reference herein). Following exposure to the lipopolysaccharide endotoxin of Gram-negitive bacteria (LPS) in an infection causing sepsis, the levels of TNFa have been shown to rise 10-20 fold to levels of up to 400 pg/ml plasma. A good correlation has been shown between serum TNFa levels and fatal outcome in infection with Gram-negative, LPS-containing meningococcal bacteria (Waage, et al., supra, 1987). Further in animal models of sepsis with subhuman primates similar increases in TNFa were noted and these changes were directly correlated with lethality (Tracey, et al., Nature, 330:662-664, 1987, incorporated by reference herein).

A prophylactically effective daily dosage of Vpr protein and/or functional fragments thereof can be about 0.1 to 100 milligrams per kilogram of body weight. Ordinarily 0.5 to 50, and preferably 1 to 10 milligrams per kilogram body weight per day. Nucleic acids encoding Vpr protein and/or functional fragments thereof can be coadministered as described above. A prophylactically effective dose of the nucleic acid encoding the Vpr protein and/or functional fragments thereof is a dose of about 1 ng to 10 mg of nucleic acid; in some embodiments, about 0.1 to about 2000 micrograms of DNA. In some preferred embodiments, therapeutically active dose is about 1 to about 1000 micrograms of DNA. In some preferred embodiments, therapeutically active dose is about 1 to about 500 micrograms of DNA. In some preferred embodiments, therapeutically active dose is about 25 to about 250 micrograms of DNA. Most preferably, therapeutically active dose is about 100 micrograms DNA.

The dosage regimen for preventing SIRS/sepsis in an individual may comprise administering one or more nucleic acid encoding the Vpr protein and/or functional fragments thereof, and Vpr protein and/or functional fragments thereof in one or more doses. The daily dosage of Vpr maybe given in divided doses 1 to 6 times a day or in sustained release form is effective to obtain desired results. The administration of Vpr proteins and/or functional fragments thereof, and/or nucleic acids that encode Vpr and/or functional fragments thereof may be repeated one to six times a day, for one to several days or weeks, or until the factors leading to the risk of developing SIRS/sepsis are reduced. In preferred embodiment, the Vpr protein and/or functional fragments thereof, and/or nucleic acids encoding them are administered by continuous infusion of the Vpr protein. In another preferred embodiment, the Vpr protein and/or functional fragments thereof, and/or nucleic acids encoding them are administered in a bolus dose, followed by continuous infusion of the Vpr protein and/or functional fragments thereof, and/or nucleic acids encoding them.

In other embodiments, the method to prevent SIRS/sepsis comprises the additional step of administering a therapeutically effective amount of an anti-infective agent, typically a aminoglycoside such as amikacin, tobramycin, netilmicin, and gentamicin, cephalosporins such as ceftazidime, related beta-lactam agents such as maxalactam, carbopenems such as imipenem, monobactam agents such as aztreonam; ampicillin and broad-spectrum penicillins, (e.g., penicillinase-resistant penicillins, ureidopenicillins or antipseudomonal penicillin or Augmentin) that are active against P. aeruginosa, Enterobacter species, indole-positive Proteus species, and Serratia. Also included within the definition of anti-infective agents are antifungal agents, amphotericin and the like, as well as anti-viral agents such as famvir and acyclovir.

The phrase “therapeutic amount of an anti-infective agent” is used herein to mean an amount sufficient to achieve a bacterial or fungal killing blood concentration, or an amount sufficient to slow the replication of a virus in the individual receiving the treatment. The therapeutic amount of anti-infective agents generally recognized as safe for administration to humans is an amount well known in the art and varies, as is also well known, with the antibiotic, fungicide, or anti-viral agent, and the type of infection being treated. Antibiotics useful in practicing the present invention include those antibiotic, antibacterial and antiseptic agents having formulations described in the Physicians' Desk Reference, Huff, B. B. ed., Medical Economics Company, Inc., Oradell, N.J. (1989), which is incorporated by reference herein.

Additionally, various agents in the treatment of SIRS/septic also may be useful in combination with the components of this invention. They include sympathomimetic amines (vasopressors) such as norepinephrine, epinephrine, isoproterenol, dopamine, and dobutamine; anti-inflammatory agents such as methylprednisolone, indomethacin and phenylbutazone; and corticosteroids such as betamethasone, hydrocortisone, methylprednisolone, or dexamethasone; anti-coagulants such as heparin, anti-thrombin III or coumarin type drugs for certain conditions and schedules; diuretics such as furosemide or ethacrynic acid; and antagonist of opiates and beta-endorphins such as naloxone; additional antagonists of tumor necrosis factor or of interleukin-1; phenothiazines; anti-histamines; glucagon; a-adrenergic blocking agents, vasodilators; plasma expanders; packed red blood cells; platelets; cryoprecipitates; fresh frozen plasma; bacterial permeability protein; clindamycin; and antibodies to (lipid A), the J5 mutant of E. coli or to endotoxin core glycolipids. Methods for preparing such adjunctive agents are widely described in the literature.

The anti-infective agent can be administered simultaneously with the Vpr composition, or independently. In a preferred embodiment, the anti-infective agent is administered with Vpr or fragment thereof. One or more adjunctive agent used to treat SIRS/sepsis agents can also be administered with Vpr protein or functional fragment thereof, and/or nucleic acid encoding Vpr or the functional fragment thereof. The adjunctive agent(s) can be administered independently or in the same formulation as the Vpr protein and/or Vpr encoding nucleic acid. In a preferred embodiment, the adjunctive agent(s) is administered with the Vpr protein or functional fragments thereof and/or nucleic acid encoding Vpr or functional fragments thereof.

B. Methods to Treat SIRS/Sepsis

Another aspect of the invention provides a therapeutic method to treat an individual who has been diagnosed as having SIRS. In a preferred embodiment, the individual has been diagnosed as having sepsis. According to the invention, methods of treating an individual with an excessive systemic immune response comprise administering to the individual the Vpr protein, as Vpr protein or a functional fragment thereof, or a nucleic acid encoding Vpr protein or a functional fragment thereof, or a combination of two or more of the same. When a nucleic acid encoding Vpr protein or a functional fragment thereof is delivered to an individual, the coding sequence is operably linked to regulatory elements. Once delivered to the individual, the nucleic acid encoding the Vpr protein is expressed and the Vpr protein is synthesized within the individual. In some embodiments, the Vpr is delivered as a nucleic acid molecule with the coding sequence for Vpr protein and/or a functional fragment thereof. In some embodiments, the Vpr and/or a functional fragment thereof is delivered as a protein. Once delivered to the individual, the presence of the Vpr protein, either delivered as a protein or produced by the expression of the nucleic acid molecule that encodes it, inhibits or down modulated the undesirable immune response.

In another embodiment, the method to treat SIRS/sepsis comprises the additional steps of monitoring the concentration of pro-inflammatory cytokines and sepsis marker proteins/conditions in the blood plasma of the individual, determining if the monitored cytokine/marker indicates the individual has SIRS/sepsis and administering additional doses of Vpr protein and/or functional fragments thereof, and/or nucleic acids encoding the Vpr protein and/or fragment thereof. Pro-inflammatory cytokines and sepsis marker proteins/conditions suitable for monitoring include, but are not limited to, cytokines such as TNFa, IL-1, IL-6, IL-8, IL-12; chemokines such as IL-8, PBP, β-TG, NAP-2, GROa, GROβ, GRO?, IP-10, SDF-1, MIP-1 a, MIP-13, MCP-1, RANTES, eotaxin, lymphotactin, fratalkine; and marker proteins/conditions such as Azurin, E. coli and other Gram-negitive bacteria endotoxins, oxidants produced by white blood cells, procalcitonin, leukocyte high-affinity Fc receptor (CD64); serum C-reactive protein (CRP); neurophil surface CD11b levels, and the ratio of selected unsaturated free fatty acids to saturated free fatty acid. In a preferred embodiment, the concentration of TNF-a in the blood plasma is monitored. In preferred embodiments, a individual is determined to have SIRS/sepsis when the concentration of TNF-a in the blood plasma is above about 100 pg/m 1, more preferably above about 50 pg/ml, most preferably above about 25 pg/ml.

An individual may be diagnosed as having SIRS/sepsis by clinical symptoms, such as for example, one or more of: (I) temperature>38° C. or <36° C.; (2) heart rate of >90 beats per minute; (3) respiratory rate>20 breathes per minute or PaCO₂<32 mm Hg; (4) white blood cell count>12000/cu mm, <4,000/cu mm, or >10% immature (band) forms; (5) organ dysfunction, hyperfusion, or hypertension (Bone et al., 1992, Chest 101:1644, which is incorporated by reference herein), as well as other methods well known to those in the art. An individual may be diagnosed as having sepsis by utilizing one of the many diagnostic assays know in the art, including but not limited to the following. Centocor Inc.'s immunometric assay for TNF-a (WO 90/06314, which is incorporated herein by reference) uses two antibodies to measure the level of this inflammatory mediator in a patient's blood. The assay developed by the National Aeronautics and Space Administration detects Pseudomonas bacteria in a patient's blood by extraction of Azurin, and its detection by a monoclonal antibody (U.S. Pat. No. 5,210,019, which is incorporated herein by reference). The sepsis assay from BioWhittaker (Walkerville, Md.) and the Limulus Amebocyte Lysate Assay form Seikagaku Kyoto Ltd. (Tokyo, Japan) measure levels of E. coli endotoxin in a patient=s blood. Other sepsis diagnostic assays detect the presence of oxidants produced by white blood cells during the septic response (U.S. Pat. No. 5,804,370, which is incorporated herein by reference), the sepsis marker peptide procalcitonin (U.S. Pat. No. 5,639,617, which is incorporated herein by reference), leukocyte high-affinity Fc receptor (CD64) expression (Davis et al., 1995, Laboratory Hematology 1:3, which is incorporated herein by reference); serum C-reactive protein (CRP) (Drews et al., 1995, Ann. NY Acad. Sci. 762:398; Kawamura and Nishida, 1995, Acta Paediatr. 84:10 which are both incorporated herein by reference); neurophil surface CD11b levels (U.S. Pat. No. 6,077,665, which is incorporated herein by reference), and the ratio of selected unsaturated free fatty acids to saturated free fatty acids (U.S. Pat. No. 5,780,237, which is incorporated herein by reference).

The phrase “therapeutically effective amount” is used herein to mean an amount sufficient to ameliorate or reduce the severity of the symptoms associated with sepsis or SIRS. Generally, a therapeutically effect amount will preferably reduce by at least about 30 percent, more preferably by at least 50 percent, most preferably by at least 90 percent, a clinically significant increase in the blood plasma level of TNFa. A clinically significant increase in the plasma level of TNFa is an increase to above about 25 pg/ml blood plasma, preferably above about 50 pg/ml blood plasma, most preferably above about 100 pg/ml blood plasma. Methods for determining the plasma TNFa levels are well known in the art, (U.S. Pat. No. 6,168,790; U.S. Pat. No. 6,063,764; Creasey et al., 1991, Circ. Shock 33:84-9; which are incorporated by reference herein).

It should be noted that levels of TNFa in normal healthy humans or in laboratory animals are estimated to be no more than about 10 pg/ml (Michie, et al., New Eng J. Med., 318:1481-1486, 1988; Mathison, et al., J. Clin. Invest., 81:1925, 1988; and Waage, et al., Lancet, 1:355-357,1987; incorporated by reference herein). Following exposure to the lipopolysaccharide endotoxin of Gram-negitive bacteria (LPS) in an infection causing sepsis, the levels of TNFa have been shown to rise 10-20 fold to levels of up to 400 pg/ml plasma. A good correlation has been shown between serum TNFa levels and fatal outcome in infection with Gram-negative, LPS-containing meningococcal bacteria (Waage, et al., supra, 1987). Further in animal models of sepsis with subhuman primates similar increases in TNFa were noted and these changes were directly correlated with lethality (Tracey, et al., Nature, 330:662-664, 1987, incorporated by reference herein).

A therapeutically effective daily dosage of Vpr protein and/or a functional fragment thereof can be about 0.1 to 100 milligrams per kilogram of body weight. Ordinarily 0.5 to 50, and preferably 1 to 10 milligrams per kilogram body weight per day. Nucleic acids encoding Vpr protein and/or functional fragments thereof can be coadministered as described above. A therapeutically effective dose of the nucleic acid encoding the Vpr protein and/or functional fragments is about 1 ng to 10 mg of nucleic acid; in some embodiments, about 0.1 to about 2000 micrograms of DNA. In some preferred embodiments, the therapeutically effective dose is about 1 to about 1000 micrograms of DNA. In some preferred embodiments, the therapeutically effective dose is about 1 to about 500 micrograms of DNA. In some preferred embodiments, the therapeutically effective dose is about 25 to about 250 micrograms of DNA. Most preferably, the therapeutically effective dose is about 100 micrograms DNA.

The dosage regimen for preventing SIRS/sepsis in an individual may comprise administering one or more of the nucleic acid encoding the Vpr protein and/or functional fragments thereof, and/or Vpr protein and/or functional fragments thereof in one or more doses. The daily dosage of Vpr maybe given in divided doses 1 to 6 times a day or in sustained release form is effective to obtain desired results. The administration of Vpr proteins and/or functional fragments thereof, and/or nucleic acids that encode Vpr and/or functional fragments thereof may be repeated 1 to six times a day, for one to several days or weeks, or until the factors leading to the risk of developing SIRS/sepsis are reduced. In preferred embodiment, the Vpr protein and/or functional fragments thereof, and/or nucleic acids encoding them are administered by continuous infusion of the Vpr protein. In another preferred embodiment, the Vpr protein and/or functional fragments thereof, and/or nucleic acids encoding them are administered in a bolus dose, followed by continuous infusion of the Vpr protein and/or functional fragments thereof, and/or nucleic acids encoding them.

In other embodiments, the method to treat SIRS/sepsis comprises the additional step of administering a therapeutically effective amount of an anti-infective agent, typically a aminoglycoside such as amikacin, tobramycin, netilmicin, and gentamicin, cephalosporins such as ceftazidime, related beta-lactam agents such as maxalactam, carbopenems such as imipenem, monobactam agents such as aztreonam; ampicillin and broad-spectrum penicillins, (e.g., penicillinase-resistant penicillins, ureidopenicillins or antipseudomonal penicillin or Augmentin) that are active against P. aeruginosa, Enterobacter species, indole-positive Proteus species, and Serratia. Also included within the definition of anti-infective agents are antifungal agents, amphotericin and the like, as well as anti-viral agents such as famvir and acyclovir.

The phrase “therapeutic amount of an anti-infective agent” is used herein to mean an amount sufficient to achieve a bacterial or fungal killing blood concentration, or an amount sufficient to slow the replication of a virus in the individual receiving the treatment. The therapeutic amount of anti-infective agents generally recognized as safe for administration to humans is an amount well known in the art and varies, as is also well known, with the antibiotic, fungicide, or anti-viral agent, and the type of infection being treated. Antibiotics useful in practicing the present invention include those antibiotic, antibacterial and antiseptic agents having formulations described in the Physicians' Desk Reference, Huff, B. B. ed., Medical Economics Company, Inc., Oradell, N.J. (1989).

Additionally, various adjunctive agents in the treatment of SIRS/septic also may be useful in combination with the components of this invention. They include sympathomimetic amines (vasopressors) such as norepinephrine, epinephrine, isoproterenol, dopamine, and dobutamine; anti-inflammatory agents such as methylprednisolone, indomethacin and phenylbutazone; and corticosteroids such as betamethasone, hydrocortisone, methylprednisolone, or dexamethasone; anti-coagulants such as heparin, anti-thrombin III or coumarin type drugs for certain conditions and schedules; diuretics such as furosemide or ethacrynic acid; and antagonist of opiates and beta-endorphins such as naloxone; additional antagonists of tumor necrosis factor or of interleukin-1; phenothiazines; anti-histamines; glucagon; a-adrenergic blocking agents, vasodilators; plasma expanders; packed red blood cells; platelets; cryoprecipitates; fresh frozen plasma; bacterial permeability protein; clindamycin; and antibodies to (lipid A), the J5 mutant of E. coli or to endotoxin core glycolipids. Methods for preparing such adjunctive agents are described widely in the literature.

The antibiotic can be administered simultaneously with the Vpr protein, or independently. In a preferred embodiment, the antibiotic is administered with the Vpr protein. One or more adjunctive agents used to treat SIRS/sepsis can also be administered with Vpr or functional fragments thereof and/or nucleic acid encoding Vpr. The adjunctive agent(s) can be administered independently or in the same formulation as the Vpr protein and/or Vpr encoding nucleic acid. In a preferred embodiment, the adjunctive agent(s) is administered with the Vpr protein and/or Vpr encoding nucleic acid.

In some embodiments of the preventive and therapeutic methods of the present invention, a combination of one or more of Vpr, a functional fragment thereof, a nucleic acid encoding Vpr, or a nucleic acid encoding a functional fragment of Vpr is administered to a patient. In some embodiments, the Vpr or a functional fragment thereof is administered to the individual in the same formulation as the nucleic acid encoding Vpr. In other embodiments, the Vpr or a functional fragment thereof is administered to the individual in a separate formulation than the nucleic acid encoding Vpr.

C. Protocols for Methods of Preventing and Treating Sirs/sepsis

Compositions and methods for delivering proteins, such as Vpr, to cells by direct DNA administration have been reported using a variety of protocols. Examples of such methods are described in U.S. Pat. No. 5,593,972, U.S. Pat. No. 5,739,118, U.S. Pat. No. 5,580,859, U.S. Pat. No. 5,589,466, U.S. Pat. No. 5,703,055, U.S. Pat. No. 5,622,712, U.S. Pat. No. 5,459,127, U.S. Pat. No. 5,676,954, U.S. Pat. No. 5,614,503, and PCT Application PCT/US95/12502, which are each incorporated herein by reference. Compositions and methods for delivering proteins to cells by direct DNA administration are also described in PCT/US90/01515, PCT/US93/02338, PCT/US93/048131, and PCT/US94/00899, which are each incorporated herein by reference. In addition to the delivery protocols described in those applications, alternative methods of delivering DNA are described in U.S. Pat. Nos. 4,945,050 and 5,036,006, which are both incorporated herein by reference. Nucleic acid molecules can also be delivered using liposome-mediated DNA transfer such as that which is described in U.S. Pat. No. 4,235,871, U.S. Pat. No. 4,241,046 and U.S. Pat. No. 4,394,448, which are each incorporated herein by reference.

Formulations comprising the nucleic acid having a sequence encoding Vpr are made up according to the mode and site of administration. Such formulation is well within the skill in the art. In addition to nucleic acids and optionally polypeptides, the formulation may also include buffers, excipients, stabilizers, carriers and diluents.

D. Pharmaceutical Compositions to Prevent and Treat SIRS/Sepsis

The pharmaceutical composition comprising Vpr protein or a fragment thereof and a pharmaceutically acceptable carrier or diluent may be formulated by one having ordinary skill in the art with compositions selected depending upon the chosen mode of administration. Suitable pharmaceutical carriers are described in the most recent edition of Remington's Pharmaceutical Sciences, A. Osol, a standard reference text in this field which is incorporated herein by reference.

For parenteral administration, the Vpr protein or fragments thereof can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions comprising Vpr protein, or fragments thereof may be administered by any means that enables the active agent to reach the agent's site of action in the body of a mammal. Because proteins are subject to being digested when administered orally, parenteral administration, i.e., intravenous, subcutaneous, intramuscular, would ordinarily be used to optimize absorption.

The dosage administered varies depending upon factors such as: pharmacodynamic characteristics; its mode and route of administration; age, health, and weight of the recipient; nature and extent of symptoms; kind of concurrent treatment; and frequency of treatment. Usually, a daily dosage of Vpr protein can be about 0.1 to 100 milligrams per kilogram of body weight. Ordinarily 0.5 to 50, and preferably 1 to 10 milligrams per kilogram per day given in divided doses 1 to 6 times a day or in sustained release form or continuous administration is effective to obtain desired results.

Another aspect of the present invention relates to pharmaceutical compositions that comprise a nucleic acid molecule that encodes Vpr and a pharmaceutically acceptable carrier or diluent. According to the present invention, genetic material that encodes Vpr protein is delivered to an individual in an expressible form. The genetic material, DNA or RNA, is taken up by the cells of the individual and expressed. Vpr that is thereby produced can inhibit immune responses, either those directed at an immunogenic vector or another undesirable immune response such as those associated with autoimmune and inflammatory disease and conditions and transplantation procedures. Thus, pharmaceutical compositions comprising genetic material that encodes Vpr are useful in the same manner as pharmaceutical compositions comprising Vpr protein.

Nucleotide sequences that encode Vpr protein operably linked to regulatory elements necessary for expression in the individual's cell may be delivered as pharmaceutical compositions using a number of strategies which include, but are not limited to, either viral vectors such as adenovirus or retrovirus vectors or direct nucleic acid transfer. Methods of delivery nucleic acids encoding proteins of interest using viral vectors are widely reported. A recombinant viral vector such as a retrovirus vector or adenovirus vector is prepared using routine methods and starting materials. The recombinant viral vector comprises a nucleotide sequence that encodes Vpr. Such a vector is combined with a pharmaceutically acceptable carrier or diluent. The resulting pharmaceutical preparation may be administered to an individual. Once an individual is infected with the viral vector, Vpr is produced in the infected cells.

Alternatively, a molecule which comprises a nucleotide sequence that encodes Vpr can be administered as a pharmaceutical composition without the use of infectious vectors. The nucleic acid molecule may be DNA or RNA, preferably DNA. The DNA molecule may be linear or circular, it is preferably a plasmid. The nucleic acid molecule is combined with a pharmaceutically acceptable carrier or diluent.

According to the invention, the pharmaceutical composition comprising a nucleic acid sequence that encodes Vpr protein may be administered directly into the individual or delivered ex vivo into removed cells of the individual which are reimplanted after administration. By either route, the genetic material is introduced into cells which are present in the body of the individual. Preferred routes of administration include intramuscular, intraperitoneal, intradermal and subcutaneous injection. Alternatively, the pharmaceutical composition may be introduced by various means into cells that are removed from the individual. Such means include, for example, transfection, electroporation and microprojectile bombardment. After the nucleic acid molecule is taken up by the cells, they are reimplanted into the individual.

The pharmaceutical compositions according to this aspect of the present invention comprise about 1 ng to 10 mg of nucleic acid in the formulation; in some embodiments, about 0.1 to about 2000 micrograms of DNA. In some preferred embodiments, the pharmaceutical compositions contain about 1 to about 1000 micrograms of DNA. In some preferred embodiments, the pharmaceutical compositions contain about 1 to about 500 micrograms of DNA. In some preferred embodiments, the pharmaceutical compositions contain about 25 to about 250 micrograms of DNA. Most preferably, the pharmaceutical compositions contain about 100 micrograms DNA.

The pharmaceutical compositions according to this aspect of the present invention are formulated according to the mode of administration to be used. One having ordinary skill in the art can readily formulate a nucleic acid molecule that encodes Vpr. In cases where injection is the chosen mode of administration, a sterile, isotonic, non-pyrogenic formulation is used. Generally, additives for isotonicity can include sodium chloride, dextrose, mannitol, sorbitol and lactose. Isotonic solutions such as phosphate buffered saline are preferred. Stabilizers include gelatin and albumin.

Regulatory elements for nucleic acid expression include promoters, initiation codons, stop codons, and polyadenylation signals. It is necessary that these regulatory elements be operably linked to the sequence that encodes the desired polypeptides and optionally the Vpr polypeptide and that the regulatory elements are operable in the individual to whom the nucleic acids are administered. For example, the initiation and termination codons must be in frame with the coding sequence. Promoters and polyadenylation signals used must also be functional within the cells of the individual.

Examples of promoters useful to practice the present invention include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

Examples of polyadenylation signals useful to practice the present invention include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In other embodiments, the pharmaceutic composition comprises a therapeutically effective amount of an anti-infective agent, typically a aminoglycoside such as amikacin, tobramycin, netilmicin, and gentamicin, cephalosporins such as ceftazidime, related beta-lactam agents such as maxalactam, carbopenems such as imipenem, monobactam agents such as aztreonam; ampicillin and broad-spectrum penicillins, (e.g., penicillinase-resistant penicillins, ureidopenicillins or antipseudomonal penicillin or Augmentin) that are active against P. aeruginosa, Enterobacter species, indole-positive Proteus species, and Serratia. Also included within the definition of anti-infective agents are antifungal agents, amphotericin and the like as well as anti-viral agents such as famvir and acyclovir.

The phrase “therapeutic amount of an anti-infective agent” is used herein to mean an amount sufficient to achieve a bacterial or fungal killing blood concentration, or an amount sufficient to slow the replication of a virus in the patient receiving the treatment. The therapeutic amount of anti-infective agents generally recognized as safe for administration to humans is an amount well known in the art and varies, as is also well known, with the antibiotic, fungicide, or anti-viral agent, and the type of infection being treated. Antibiotics useful in practicing the present invention include those antibiotic, antibacterial and antiseptic agents having formulations described in the Physicians' Desk Reference, Huff, B. B. ed., Medical Economics Company, Inc., Oradell, N.J. (1989).

Additionally, various adjunctive agents in the treatment of SIRS/sepsis also may be useful in the pharaceutical composition of this invention. They include sympathomimetic amines (vasopressors) such as norepinephrine, epinephrine, isoproterenol, dopamine, and dobutamine; anti-inflammatory agents such as methylprednisolone anti-inflammatory agents such as indomethacin and phenylbutazone; and corticosteroids such as betamethasone, hydrocortisone, methylprednisolone, or dexamethasone; anti-coagulants such as heparin, anti-thrombin III or coumarin type drugs for certain conditions and schedules; diuretics such as furosemide or ethacrynic acid; and antagonist of opiates and beta-endorphins such as naloxone; additional antagonists of tumor necrosis factor or of interleukin-1; phenothiazines; anti-histamines; glucagon; a-adrenergic blocking agents, vasodilators; plasma expanders; packed red blood cells; platelets; cryoprecipitates; fresh frozen plasma; bacterial permeability protein; clindamycin; and antibodies to (lipid A), the J5 mutant of E. coli or to endotoxin core glycolipids. Methods for preparing such adjunctive agents are described widely in the literature.

The foregoing embodiments are meant to illustrate the invention and are not to be construed to limit the invention in any way. Those skilled in the art will recognize modifications that are within the spirit and scope of the invention. All references cited herein are hereby incorporated by reference in their entirety.

1. A method of treating an individual who has been diagnosed as having SIRS or sepsis comprising the step of administering to said individual a therapeutically effective amount of an immuno-modulating pharmaceutical compositions comprising one or more of the components selected from the group consisting of:
i) Vpr protein;
ii) a function fragment of Vpr protein;
iii) a nucleic acid encoding Vpr protein operably linked to regulatory elements; and
iv) a nucleic acid encoding a functional fragment of Vpr protein operably linked to regulatory elements.

2. The method of claim 1 wherein the individual is administered a nucleic acid encoding Vpr protein or functional fragment thereof operably linked to regulatory elements.

3. The method of claim 2, wherein the nucleic acid is administered in a dose of 1 to 500 micrograms nucleic acid.

4. The method of claim 2, wherein the nucleic acid is administered in a dose of 25 to 250 micrograms nucleic acid.

5. The method of claim 2, wherein the nucleic acid is administered in a dose of about 100 micrograms nucleic acid.

6. The method of claim 2, wherein the nucleic acid encoding Vpr protein or functional fragment thereof operably linked to regulatory elements is contained in a plasmid.

7. The method of claim 2, wherein the nucleic acid encoding Vpr protein or functional fragment thereof operably linked to regulatory elements is contained in a viral vector.

8. The method of claim 7, wherein the viral vector is selected from the group consisting of a retroviral vector and an adenoviral vector.

9. The method of claim 1, wherein the step of administering the immunomodulating pharmaceutical composition is repeated at least once.

10. The method of claim 9, wherein the step of administering the immunomodulating pharmaceutical composition is undertaken 1 to 6 times a day.

11. The method of claim 1, which additionally comprises at least one step of administering to said individual a therapeutically effective amount of an anti-infective agent.

12. The method of claim 11, wherein the anti-infective agent is selected from the group consisting of: amikacin, tobramycin, netilmicin, gentamicin, cephalosporin, ceftazidime, maxalactam, carbopenem, imipenem, aztreonam, ampicillin, penicillin, ureidopenicillin, augmentin, amphotericin, famvir and acyclovir.

13. The method of claim 11, wherein the step of administering the anti-infective agent is performed at the same time as the step of administering the immunomodulating pharmaceutical preparation.

14. The method of claim 1, which comprises the additional steps of monitoring the concentration of pro-inflammatory cytokines and sepsis marker proteins/conditions in the blood plasma of the individual, determining the need for additional doses of the immuno-modulating pharmaceutical composition and administering additional doses of the immuno-modulating pharmaceutical composition.

15. The method of claim 14, wherein the blood plasma level of TNFa is monitored and the need for additional doses of the immuno-modulating pharmaceutical composition is determined by a blood plasma level of TNFa above about 25 pg/ml.

16. The method of claim 1, wherein the step of administering the immunomodulating pharmaceutical composition comprises continuous administration.

17. The method of claim 1 wherein the individual is administered Vpr protein of a functional fragment thereof.

18. The method of claim 17, wherein the Vpr protein or functional fragment thereof is administered at 0.1 to 100 mg/kg body weight per day.

19. The method of claim 17, wherein the Vpr protein or functional fragment thereof is administered at 0.5 to 50 mg/kg body weight per day.

20. The method of claim 17, wherein the Vpr protein or functional fragment thereof is administered at 1.0 to 10 mg/kg body weight per day.

21. A method of preventing sepsis in an individual who has been identified as being at an elevated risk of contracting sepsis comprising the step of administering to said individual a prophylactically effective amount of an immuno-modulating pharmaceutical compositions comprising one or more of the components selected from the group consisting of:
i) Vpr protein;
ii) a function fragment of Vpr protein;
iii) a nucleic acid encoding Vpr protein operably linked to regulatory elements; and
iv) a nucleic acid encoding a functional fragment of Vpr protein operably linked to regulatory elements.

22-40. (Cancelled).

41. A pharmaceutical composition useful for preventing and treating sepsis, comprising an anti-infective agent and one or more of the components selected from the group consisting of:
i) Vpr protein;
ii) a function fragment of Vpr protein;
iii) a nucleic acid encoding Vpr protein operably linked to regulatory elements; and
iv) a nucleic acid encoding a functional fragment of Vpr protein operably linked to regulatory elements.

42. The pharmaceutical composition of claim 41, wherein the anti-infective agent is selected from the group consisting of: amikacin, tobramycin, netilmicin, gentamicin, cephalosporin, ceftazidine, maxalactam, carbopenem, imipenem, aztreonam, ampicillin, penicillin, ureidopenicillin, augmentin, amphotericin, famvir and acyclovir.

43. The pharmaceutical composition of claim 40, which additionally comprises at least one adjunctive agent in the treatment of SIRS/sepsis.
